(19) United States
(12) Patent Application Publication
(10) Pub. No.: US 2004/0092793 A1
(43) Pub. Date: May 13, 2004

(54) Electronic endoscope apparatus with static electricity measures

(75) Inventor: Nobuyuki Akai, Saitama-shi (JP)

Correspondence Address: Ronald R. Snider, P.O. Box 27613, Washington, DC, 20038-7613, US

(73) Assignee: Fuji Photo Optical Co., Ltd., Saitama-shi (JP)

(21) Appl. No.: 10/694,883

(22) Filed: Oct. 29, 2003

(30) Foreign Application Priority Data
Nov. 1, 2002 (JP) 2002-319339

(51) Int. Cl. A61B 1/00

(52) U.S. Cl. 600/134

(57) ABSTRACT

Metal members for an insertion section, an operation section, and a cable section, respectively, are provided as a sheath for a scope. These metal members are electrically connected together. The metal member in the cable section is connected to a shield box in a connector circuit section via a static-electricity-suppressing part. The shield box is connected to a housing ground for a processor device via a contact piece spring. Furthermore,the metal member in the cable section may be connected to a ground terminal in the connector circuit section via a static-electricity-suppressing part. Thus, static electricity measures can be taken using only a small number of parts, while preventing the static-electricity-suppressing part from affecting the operations and characteristics of electronic circuits in the scope.

### BACKGROUND OF THE INVENTION

1. Field of the Invention

This application claims the priority of Japanese Patent Applications No. 2002-319339 filed on Nov. 1, 2002 which is incorporated herein by reference.

The present invention is an electronic endoscope apparatus, and in particular, to an arrangement for protecting electronic parts in a scope from externally applied static electricity.

2. Description of the Related Art

FIG. 3 schematically shows the configuration of an electronic endoscope apparatus for use in medical fields and the like. In this apparatus, a scope (electronic endoscope) 1 is removably connected to a processor device 2 by a connector section 3. The scope 1 is provided with a CCD circuit section 4 having a CCD (charge Coupled Device), at its leading end. The CCD circuit section 4 is driven by a signal supplied by the connector circuit section 5 via a signal line 6a. A signal indicating an image taken by the CCD circuit section 4 is supplied to the processor device 2 through a signal line 6b via the connector circuit section 5. Furthermore, a plurality of switches 7 including a freeze switch are arranged on an operation section of the scope 1 to form and record still images. An operation signal from any of the switches 7 is supplied to the processor device 2 through a signal line 6c via the connector circuit section 5.

In the apparatus shown in FIG. 3, an output signal from the CCD circuit section 4 is subjected to a predetermined process in the connector circuit section 5. The processed signal is then subjected to a color video process in the processor device 2. Then, a video for an object is displayed on a monitor 8. Furthermore, the freeze switch 7 is operated to form a still image that can be recorded in a storage device or the like.

In the above electronic endoscope apparatus, static electricity measures have been proposed in order to protect electronic parts such as ICs (Integrated Circuits) and transistors which are arranged inside the scope 1. These static electricity measures arrange static-electricity-suppressing parts for desired signal lines or power lines. For example, in FIG. 3, static-electricity-suppressing parts 9a, 9b, and 9c are arranged between a ground and the signal lines 6a, 6b, and 6c, respectively.

However, as described above, if the static-electricity-suppressing parts 9a, 9b, and 9c are connected directly to the signal lines 6a, 6b, and 6c, respectively, the impedances (electrostatic capacity and the like) of circuit elements in these parts may affect the operations and characteristics of internal electronic circuits in the CCD circuit section 4 and connector circuit section 5. Furthermore, when the static-electricity-suppressing parts (9a to 9c) are provided in association with the desired signal lines (6a to 6c) or power lines as in FIG. 3, a large number of static-electricity-suppressing parts must be installed. This disadvantageously increases the size of the scope as well as costs.

### SUMMARY OF THE INVENTION

The present invention is provided in view of the above problems. It is an object of the present invention to provide an electronic endoscope apparatus that can take effective static electricity measures using only a small number of parts, while preventing a static-electricity-suppressing part from affecting the operations and characteristics of electronic circuits in a scope.

To accomplish this object, the present invention is characterized by comprising a scope in which a solid image pickup element is mounted and in which a metal member is provided as a sheath and a processor device to which the scope is connected and which executes signal processing, and in that a static-electricity-suppressing part is provided between the sheath metal member of the scope and a processor device housing ground.

The metal member as the sheath is composed of a ring-like metal member including an angle ring in an insertion section, a metal frame in an operation section, and a ring-like metal member in a cable section, the metal frame, and the ring-like metal member being electrically connected together.

The sheath metal member of the scope can be connected to the processor device housing ground via a shield box in a scope-side connector circuit section.

Another aspect of the present invention is characterized by comprising a scope in which a solid image pickup element is mounted and in which a metal member is provided as a sheath and a processor device to which the scope is connected and which executes signal processing, and in that a static-electricity-suppressing part is provided between the sheath metal member of the scope and a scope-side circuit ground.

The scope-side circuit ground maybe a ground terminal in the scope-side connector circuit section.

With the above arrangement, the static-electricity-suppressing part is composed of a static-electricity-suppressing element or the like utilizing a surge absorber or an air gap. The static-electricity-suppressing part is arranged between the sheath metal member and the processor device housing ground, i.e. a ground for a commercial power source. Alternatively, the static-electricity-suppressing part maybe arranged between the sheath metal member and the connector circuit section ground of the scope. As a result, static electricity externally applied to the scope flows from the sheath metal member to the ground without passing through electronic circuits (parts) in the scope.

### BRIEF DESCRIPTION OF THE DRAWINGS

FIG. 1 is a diagram showing the whole configuration of an electronic endoscope apparatus according to a first embodiment of the present invention;

FIG. 2 is a diagram showing the configuration of integral parts according to a second embodiment; and

FIG. 3 is a diagram showing the configuration of a conventional electronic endoscope apparatus that takes static electricity measures.

### DETAILED DESCRIPTION OF THE PREFERRED EMBODIMENTS

First Embodiment

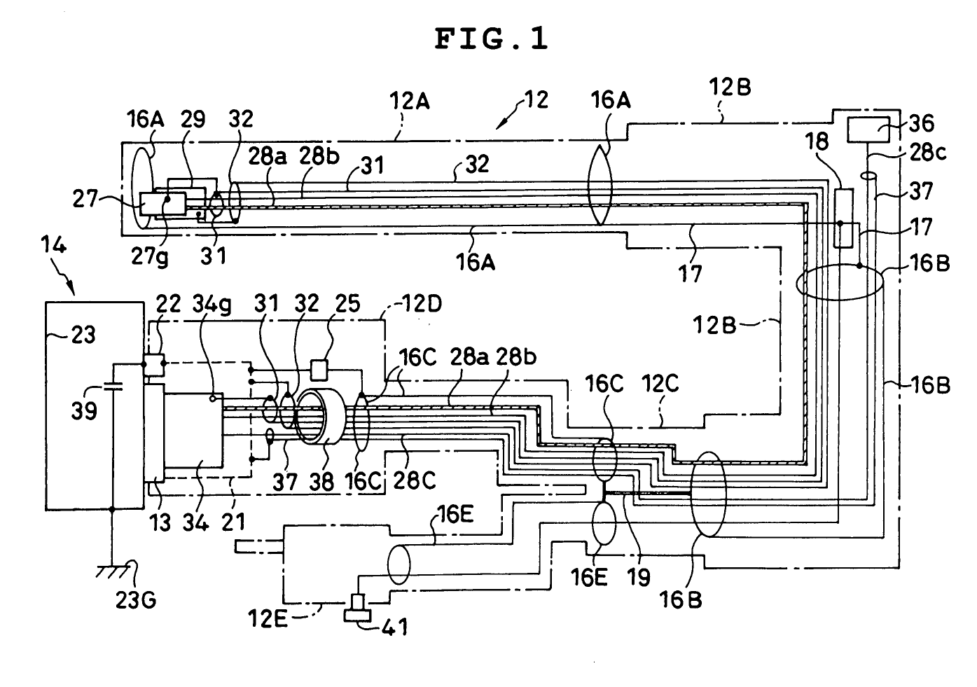

FIG. 1 shows the configuration of an electronic endoscope apparatus according to a first embodiment. As shown in this figure, a scope (electronic endoscope) 12 is removably connected to a processor device 14 by a connector 13. The scope 12 has an insertion section 12A, an operation section 12B, and a cable section 12C. The cable section 12C is bifurcated in its middle and has an electric connector section 12D and an optical connector section 12E at the ends of the respective branches.

As a sheath for the scope 12, a ring-like metal member 16A including an angle ring is arranged in the insertion section 12A. A metal member 16B such as a frame is arranged in the operation section 12B. Ring-like metal members 16C and 16E such as helical traveling-wave tubes are arranged in the cable section 12C. A coating made of a synthetic resin is formed outside the metal member 16A. In an embodiment, the insertion section metal member 16A and the operation section metal member 16B are electrically connected together via a lead (connection line) 17. The lead 17 is also connected to a terminal block 18. Furthermore, the operation section metal member 16B is electrically connected to the cable section metal members 16C and 16E via a connection terminal block 19.

Moreover, the electric connector section 12D is provided with a shield box 21 that protects internal circuits. The shield box 21 is electrically connected to a housing 23 for the processor device 14 by a contact piece spring 22. The housing 23 is connected to housing ground 23G that is a ground for a commercial power source.

A static-electricity-suppressing part 25 is attached between the shield box 21 and the cable section metal member 16C. As a result, the metal members 16A, 16B, and 16C constituting the sheaths for the insertion section 12A, operation section 12B, and cable section 12C, respectively, are all connected to the housing ground 23G via the static-electricity-suppressing part 25. The static-electricity-suppressing part 25 is composed of a generally known static-electricity-suppressing element (suppressor) utilizing a surge absorber or an air gap.

On the other hand, the insertion section 12A is provided with a CCD circuit section 27 at its leading end; a CCD and an electronic part such as a buffer circuit are mounted on the CCD circuit section 27. The CCD circuit section 27 connects to a coaxial signal line 28a and a signal line 28b having a plurality of electric wires. The outer periphery of the CCD circuit section 27 is wrapped in a shield member 29. Furthermore, the coaxial signal line 28a and the signal line 28b are covered with a double shield of, for example, a mesh-like inner and outer shields 31 and 32. The double shield (31 and 32) and the shield member 29 are provided to eliminate the adverse effects of noise. The inner shield 31 of the double shield is connected to a ground terminal 27g in the CCD circuit section 27. The outer shield 32 is connected to the shield member 29.

The electric connector section 12D is provided with a connector circuit section (for example, a setup board) 34 including a signal processing circuit or the like. The coaxial signal line 28a and the signal line 28b are connected to the connector circuit section 34. The outer periphery of the connector circuit section 34 is covered with the shield box 21 in order to prevent the adverse effects of noise. Furthermore, the double shield (31 and 32) is also extended to the electric connector section 12D. The inner shield 31 is connected to a ground (patient-side ground) terminal 34g in the connector circuit section 34. The outer shield 32 is connected to the shield box 21. The ground terminal 34g is connected to a ground line in the circuit in the processor device 14 by a ground line.

The operation section 12B is provided with a plurality of switches 36 including a freeze switch in order to form and record still images. The switches 36 are also connected to the connector circuit section 34 via a signal line 28c. A shield 37 for the signal line 28c is connected to the shield box 21. To remove noise, the electric connector section 12D is provided with a ferrite core 38 inside which the signal lines 28a to 28c and the shields 31, 32, and 37 are arranged.

Furthermore, a noise removing capacitor 39 having a withstanding voltage of 4 kV or higher is provided between the housing 23 for the processor device 14 and the ground 23G. The optical connector 12E is connected to a light source device (not shown) and is provided with an S connector 41 connected to a ground for an electric scalpel. The S connector 41 is connected to the terminal block 18 in the operation section 12B.

The first embodiment is configured as described above. The metal member 16A in the insertion section 12A is connected to the metal member 16B in the operation section 12B by a lead 17. The metal member 16B is connected to the metal member 16C in the cable section 12C by a connection terminal block 19. The metal member 16C is connected to the shield box 21 via the static-electricity-suppressing part 25. The shield box 21 is connected to the housing ground 23G for the processor device 14 via the contact piece spring 22. Consequently, the metal members 16A to 16C constituting the sheath for the scope 12 are all connected to the ground for the commercial power source via the static-electricity-suppressing part 25.

Therefore, even if external static electricity is applied to the scope 12, it can be appropriately diverted to the ground 23G. Accordingly, the electronic circuits (parts) in the scope 12 are not adversely affected by the external static electricity. Furthermore, in recent years, the scope 12 has tended to undergone an EMC (Electromagnetic Compatibility) test, and the present embodiment advantageously protects the electronic circuit during the EMC test. Moreover, the static-electricity-suppressing part is not connected directly to the plurality of signal lines compared to the prior art. This advantageously prevents the adverse effects on the operations and characteristics of the electronic parts connected to the signal lines.

In the above first embodiment, the metal member 16C in the cable section 12C is connected to the shield box 21. However, the metal member 16C may be connected directly to the contact piece spring 22 or the housing 23 for the processor device 14.

Second Embodiment

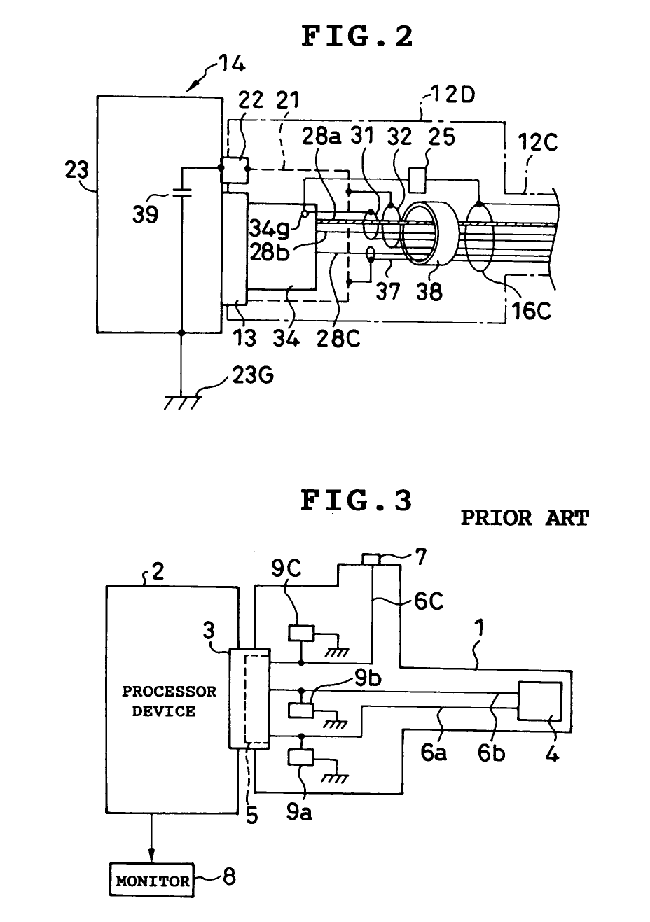

FIG. 2 shows the configuration of a second embodiment of the present invention. In the second embodiment, as shown in FIG. 2, the metal member 16C in the cable section 12C is connected to the ground terminal 34g in the connector circuit section 34 via the static-electricity-suppressing part 25. In this case, the metal members 16A, 16B, and 16C constituting the sheath for the scope 12 are connected to the ground terminal 34g, i.e. a patient-side ground, via the static-electricity-suppressing part 25. This allows static electricity to be absorbed by the ground.

As described above, according to the first and second embodiments, static electricity can be appropriately diverted to the ground while preventing the static-electricity-suppressing part from affecting the operations and characteristics of the electronic circuits in the scope. Furthermore, the static-electricity-suppressing part is not arranged for each of the plurality of signal lines. Therefore, effective static electricity measures can be taken using only a small number of parts.

What is claimed is:

1. An electronic endoscope apparatus comprising:
a scope in which a solid image pickup element is mounted and in which a metal member is provided as a sheath;
a processor device to which the scope is connected and which executes signal processing; and
wherein a static-electricity-suppressing part is provided between the sheath metal member of said scope and said processor device housing ground processor device.

2. The electronic endoscope apparatus according to claim 1, wherein said metal member as the sheath is composed of a ring-like metal member including an angle ring in an insertion section, a metal frame in an operation section, and a ring-like metal member in a cable section, the angle ring, the metal frame, and the ring-like metal member being electrically connected together.

3. The electronic endoscope apparatus according to claim 1, wherein the sheath metal member of said scope is connected to said processor device housing ground via a shield box in a scope-side connector circuit section.

4. An electronic endoscope apparatus comprising:
a scope in which a solid image pickup element is mounted and in which a metal member is provided as a sheath and;
a processor device to which the scope is connected and which executes signal processing; and
wherein a static-electricity-suppressing part is provided between the sheath metal member of said scope and a scope-side circuit ground.

5. The electronic endoscope apparatus according to claim 4, wherein said metal member as the sheath is composed of a ring-like metal member including an angle ring in an insertion section, a metal frame in an operation section, and a ring-like metal member in a cable section, the angle ring, the metal frame, and the ring-like metal member being electrically connected together.

6. The electronic endoscope apparatus according to claim 4, wherein said scope-side circuit ground is a ground terminal in the scope-side connector circuit section.
